(19) United States
(12) Patent Application Publication
(10) Pub. No.: US 2022/0310212 A1
(43) Pub. Date: Sep. 29, 2022

(54) CALCULATION METHOD, CALCULATOR SYSTEM, AND CALCULATOR

(71) Applicant: JSR CORPORATION, Tokyo (JP)

(72) Inventors: Junta Fuchiwaki, Tokyo (JP); Yuya Onishi, Tokyo (JP); Yoshio Takimoto, Tokyo (JP)

(73) Assignee: JSR CORPORATION, Tokyo (JP)

(21) Appl. No.: 17/807,079

(22) Filed: Jun. 15, 2022

Related U.S. Application Data
(63) Continuation of application No. PCT/JP2020/046819, filed on Dec. 15, 2020.

(30) Foreign Application Priority Data
Dec. 19, 2019 (JP) 2019-228865

(51) Int. Cl. G16C 10/00; G06N 10/60

(52) U.S. Cl. CPC G16C 10/00; G06N 10/60

(57) ABSTRACT

A method is implemented to select a calculator for performing given processing using a quantum algorithm or a combined algorithm of a classical algorithm and the quantum algorithm. The method comprises a calculation operation, a selection operation, and a control operation. The calculation operation calculates a quantum bit or a quantum volume for performing the given processing using the quantum algorithm, or for a portion of the quantum algorithm when performing the given processing using the combined algorithm. The selection operation selects a calculator for performing the given processing based on the quantum bit or the quantum volume. The control operation generates a control signal to be transmitted to the quantum calculator when the selected calculator includes a quantum calculator. The control signal may correspond to an instruction that initiates the quantum calculator to start the quantum algorithm.

### CROSS-REFERENCE TO RELATED APPLICATION

This application is a continuation of PCT International Application No. PCT/JP2020/046819 filed on Dec. 15, 2020 which claims the benefit of priority from Japanese Patent Application No. 2019-228865 filed on Dec. 19, 2019, the entire contents of which are incorporated herein by reference.

### BACKGROUND OF THE INVENTION

### 1. Field of the Invention

One or more embodiments described herein relate to a calculation method, a calculator system, and a calculator.

### 2. Description of the Related Art

A quantum calculator performs calculations using a calculation paradigm different from a classical calculator. A quantum calculator causes a unitary matrix to act on a quantum state (such as a quantum entangled state, for example) that is initially generated under a predetermined quantum algorithm. Examples of such an algorithm include Shore's prime factorization algorithm and Grover's search algorithm. A calculation result is then determined based on the generated quantum state.

Simulation and execution environments of quantum calculation are provided as cloud services. Examples include Azure (registered trademark) Quantum, AWS (registered trademark) Braket, and IBM Q-Experience (registered trademark). In these cloud services, for example, a test and simulation environment for a quantum algorithm on a classical computer is provided. A created quantum algorithm can then be executed on quantum hardware, such as a gate-type superconducting computer, a quantum annealing superconducting computer, or an ion trap computer.

Differences between a quantum calculator and a classical calculator may be understood in the example case of performing a molecular orbital calculation. An increasing calculation speed of the quantum calculator (with respect to the number of atoms N) is gentle compared with a classical calculator. However, a quantum calculator having a large number of quantum bits is difficult to make. As a result, the performance of a quantum calculator may be limited by the number of quantum bits. In view of these considerations, it may be desirable to select one of the classical calculator or the quantum calculator to perform calculations depending on the amount of processing that is to be performed.

In other cases, a combined algorithm may perform calculations using both a classical calculator and a quantum calculator. For example, Variational Quantum Eigensolver (VQE) is an algorithm for measuring an expected value of Hamiltonian after generating a predetermined trial wave function. The quantum calculator may generate the predetermined trial wave function based on a calculus of variations. Processing may then be performed by a classical calculator based on a measurement result, to embed the quantum calculator in the classical calculator based on the calculus of variations.

In another case, calculations may be performed using a plurality of quantum calculators which have quantum bits that are different.

### SUMMARY OF THE INVENTION

In accordance with some embodiments described herein, an improved method, calculator system, and calculator is provided which at least partially overcomes problems of other calculators.

In accordance with one or more embodiments, a method is executed by a classical calculator that performs processing of selecting a calculator for performing given processing in a case of performing the given processing using a quantum algorithm or a combined algorithm of a classical algorithm and the quantum algorithm. The method includes performing:

a calculation operation to calculate a required quantum bit or a required quantum volume for performing the given processing using the quantum algorithm, or required for a portion of the quantum algorithm in a case of performing the given processing by using the combined algorithm;

a selection operation to select the calculator for performing the given processing based on the quantum bit or the required quantum volume calculated at the calculation operation; and

a control operation to generate, in a case in which the calculator selected at the selection operation includes a quantum calculator, a control signal to be transmitted to the quantum calculator, the control signal being related to an instruction by which the quantum calculator starts the quantum algorithm.

In accordance with one or more other embodiments, a calculator system comprises a plurality of calculators and a classical calculator that performs processing of selecting a calculator for performing given processing from among the calculators in a case of performing the given processing using a quantum algorithm or a combined algorithm of a classical algorithm and the quantum algorithm. The calculators may include at least one quantum calculator, and the classical calculator may comprise:

a calculation unit (or calculator) configured to calculate a required quantum bit or required quantum volume for performing the given processing by using the quantum algorithm, or required for a portion of the quantum algorithm in a case of performing the given processing by using the combined algorithm;

a sorting unit (or sorter) configured to select the calculator for performing the given processing based on the quantum bit or the required quantum volume calculated by the calculation unit; and

a transmission unit (or transmitter) configured to transmit, in a case in which the calculator selected by the sorting unit includes the quantum calculator, an instruction by which the quantum calculator starts the quantum algorithm to the quantum calculator.

In accordance with one or more embodiments, a classical calculator is configured to perform processing of selecting a calculator for performing given processing from among a plurality of calculators in a case of performing the given processing using a quantum algorithm or a combined algorithm of a classical algorithm and the quantum algorithm. The classical calculator may comprise:

a calculation unit (or calculator or calculation logic) configured to calculate a required quantum bit or required quantum volume for performing the given processing using the quantum algorithm, or required for a portion of the quantum algorithm in a case of performing the given processing by using the combined algorithm;

a sorting unit (or sorter) configured to select the calculator for performing the given processing based on the quantum bit or the required quantum volume calculated by the calculation unit; and

a transmission unit (or transmitter) configured to transmit, in a case in which the calculator selected by the sorting unit includes a quantum calculator, an instruction by which the quantum calculator starts the quantum algorithm to the quantum calculator.

The above and other objects, features, advantages and technical and industrial significance of this invention will be better understood by reading the following detailed description of presently preferred embodiments of the invention, when considered in connection with the accompanying drawings.

The following embodiments are disclosed:

1. A method executed by a classical calculator to perform processing of selecting a calculator for performing given processing in a case of performing the given processing by using a quantum algorithm or a combined algorithm of a classical algorithm and the quantum algorithm, the method including performing:

a calculation step to calculate a required quantum bit or a required quantum volume required for performing the given processing by using the quantum algorithm, or required for a portion of the quantum algorithm in a case of performing the given processing by using the combined algorithm;

a selection step to select the calculator for performing the given processing based on the quantum bit or the required quantum volume calculated at the calculation step; and

a control step to generate, in a case in which the calculator selected at the selection step includes a quantum calculator, a control signal to be transmitted to the quantum calculator, the control signal being related to an instruction by which the quantum calculator starts the quantum algorithm.

2. The method according to embodiment 1, wherein, at the control step, in a case in which the calculator selected at the selection step includes a quantum calculator, the quantum calculator generates, as the control signal, information required for the quantum calculator to generate a quantum state to be generated at the time of starting the quantum algorithm.

3. The method according to embodiment 1, wherein the given processing is processing of performing quantum chemistry calculation on a given chemical structure.

4. The method according to embodiment 3, wherein the given chemical structure is a chemical structure read out from a storage unit storing a list of chemical structures.

5. The method according to embodiment 1, wherein,

at the calculation step, in a case in which the given processing is constituted of a plurality of small pieces of processing, the quantum bit or the required quantum volume is calculated for each of the small pieces of processing,

at the selection step, in a case in which the given processing is constituted of a plurality of small pieces of processing, the calculator is selected for each of the small pieces of processing, and

at the control step, in a case in which the given processing is constituted of a plurality of small pieces of processing, the control signal is generated for each of the small pieces of processing.

6. The method according to embodiment 1, further performing:

a division determination step to determine a division method for dividing the given processing into a plurality of small pieces of processing; and

a division step to divide the given processing into the small pieces of processing based on the determined division method, wherein,

at the selection step, the calculator is selected for each of the small pieces of processing divided at the division step, and

at the control step, the control signal is generated for each of the small pieces of processing divided at the division step.

7. The method according to embodiment 6, wherein, at the selection step, the calculator is selected in accordance with the quantum bit or the required quantum volume calculated at the calculation step, and at least one of capacity, a characteristic, and a vacant state of an available calculator resource.

8. The method according to embodiment 7, wherein, at the selection step, an execution timing or execution order for executing the calculator is further selected in accordance with the quantum bit or the required quantum volume calculated at the calculation step, and at least one of capacity, a characteristic, and a vacant state of an available calculator resource.

9. The method according to embodiment 1, wherein, at the selection step, the calculator is selected so that the quantum bit does not exceed a quantum bit that is able to be used by the calculator, or the required quantum volume does not exceed a required quantum volume of the calculator.

10. The method according to embodiment 1, further performing:

a display control step to cause a display unit to display the quantum bit or the required quantum volume calculated at the calculation step.

11. The method according to embodiment 1, further performing:

a generation step to generate a list of calculators capable of performing the processing based on the quantum bit or the required quantum volume calculated at the calculation step; and

a display control step to cause a display unit to display the list of calculators capable of performing the processing generated at the generation step.

12. The method according to embodiment 10, wherein the display unit is disposed on a terminal that is different from a terminal on a provider side that is a terminal on which the classical calculator is disposed, and is a user terminal that is a terminal being able to be operated by a user.

13. The method according to embodiment 1, wherein, at the selection step, a calculator for performing the processing is selected from both of the classical calculator and the quantum calculator based on the quantum bit or the required quantum volume.

14. The method according to embodiment 1, wherein the combined algorithm is a variational quantum eigensolver (VQE) method.

15. A calculator system comprising a plurality of calculators and a classical calculator that performs processing of selecting a calculator for performing given processing from among the calculators in a case of performing the given processing by using a quantum algorithm or a combined algorithm of a classical algorithm and the quantum algorithm, wherein

the calculators include at least one quantum calculator,

the classical calculator comprises:
a calculation unit configured to calculate a required quantum bit or required quantum volume required for performing the given processing by using the quantum algorithm, or required for a portion of the quantum algorithm in a case of performing the given processing by using the combined algorithm;
a sorting unit configured to select the calculator for performing the given processing based on the quantum bit or the required quantum volume calculated by the calculation unit; and
a transmission unit configured to transmit, in a case in which the calculator selected by the sorting unit includes the quantum calculator, an instruction by which the quantum calculator starts the quantum algorithm to the quantum calculator.

16. The calculator system according to embodiment 15, comprising:

a function of transmitting the quantum bit or the required quantum volume calculated by the calculation unit, or information related to the calculator selected by the sorting unit.

17. The calculator system according to embodiment 15, further comprising:

a display unit configured to display, to a user, the quantum bit or the required quantum volume calculated by the calculation unit, or information related to the calculator selected by the sorting unit.

18. The calculator system according to embodiment 17, further comprising:

a display unit configured to display, to the user, the quantum bit or the required quantum volume calculated by the calculation unit, or the information related to the calculator selected by the sorting unit, and a character string representing a structural formula or atomic coordinates or a chemical structure, or information specifying a compound.

19. The calculator system according to embodiment 15, wherein the classical calculator comprises a first classical calculator having a function of the calculation unit, and a second classical calculator that is connected to the first classical calculator via a network and has functions of the sorting unit and the transmission unit.

20. A classical calculator configured to perform processing of selecting a calculator for performing given processing from among a plurality of calculators in a case of performing the given processing by using a quantum algorithm or a combined algorithm of a classical algorithm and the quantum algorithm, the classical calculator comprising:

a calculation unit configured to calculate a required quantum bit or required quantum volume required for performing the given processing by using the quantum algorithm, or required for a portion of the quantum algorithm in a case of performing the given processing by using the combined algorithm;

a sorting unit configured to select the calculator for performing the given processing based on the quantum bit or the required quantum volume calculated by the calculation unit; and

a transmission unit configured to transmit, in a case in which the calculator selected by the sorting unit includes a quantum calculator, an instruction by which the quantum calculator starts the quantum algorithm to the quantum calculator.

### BRIEF DESCRIPTION OF THE DRAWINGS

FIG. 1 is a diagram representing an outline of a calculator system according to an embodiment of the present invention;

FIG. 2A is a diagram for explaining a background according to the embodiment;

FIG. 2B is a diagram for explaining a background according to the embodiment;

FIG. 3 is a flowchart illustrating a processing procedure performed by a computer program according to a first embodiment;

FIG. 4 is a flowchart illustrating a processing procedure performed by a computer program according to a first modification of the first embodiment;

FIG. 5 is a flowchart illustrating a processing procedure performed by a computer program according to a second embodiment; and

FIG. 6 is a flowchart for specifically explaining processing at Operation S200 in FIG. 5.

### DETAILED DESCRIPTION OF THE PREFERRED EMBODIMENTS

The following describes a computer program, a calculator system, and a calculator according to embodiments of the present invention with reference to the attached drawings.

### First Embodiment

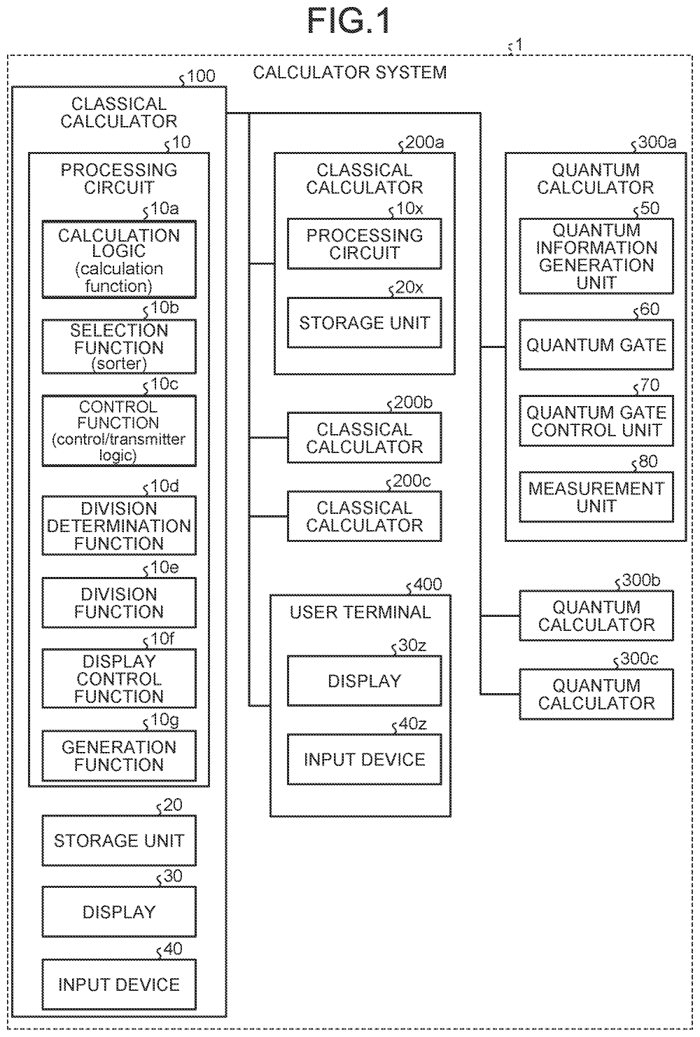

FIG. 1 is a diagram representing a calculator system 1 according to an embodiment of the present invention. The calculator system 1 includes a classical calculator 100 for controlling other calculators, classical calculators 200a, 200b, 200c . . . for performing calculations without using quantum calculation, and quantum calculators 300a, 300b, 300c . . . for performing calculation using quantum calculation. Herein, each of the classical calculators 100, 200a, 200b, and 200c is a calculator in a normal sense that can be represented as a Turing machine, for example, a von Neumann computer.

The calculator system 1 may include two or more of calculators including the classical calculators 200a, 200b, 200c . . . and the quantum calculators 300a, 300b, 300c . . . . The calculator system 1 may include only the quantum calculator, or may include both of the classical calculator and the quantum calculator.

The classical calculator 100 is a calculator for controlling the other calculators (the classical calculators 200a, 200b, 200c . . . , the quantum calculators 300a, 300b, and 300c), and includes a processing circuit 10, a storage unit 20, a display 30, and an input device 40. The processing circuit 10 includes a calculation function 10a, a selection function 10b, a control function (or control logic) 10c, a division determination function 10d, a division function 10e, a display control function 10f, and a generation function 10g.

In one embodiment, the processing circuit 10 may include a processor that implements functions such as the calculation function 10a and the selection function 10b by reading out, from the storage unit 20, and executing a computer program. The processing unit 10 may include, for example, one or more of a central processing unit (CPU), a graphical processing unit (GPU), an application specific integrated circuit (ASIC), a programmable logic device (PLD), or the like. In one embodiment, processing functions of the calculation function 10a, the selection function 10b, the control function 10c, the division determination function 10d, the division function 10e, the display control function 10f, the generation function 10g, and the like are stored in the storage unit 20 in a form of a computer program that can be executed by the processor.

Accordingly, the processing circuit 10 that has read out the computer program may perform the calculation function 10a, the selection function 10b, the control function 10c, the division determination function 10d, the division function 10e, the display control function 10f, and the generation function 10g. In one embodiment, the processing circuit 10 may execute the computer program (or instructions) to cause the classical calculator 100 to perform a calculation operation by the calculation function 10a, perform a selection operation by the selection function 10b, perform a control operation by the control function 10c, perform a division determination operation by the division determination function 10d, perform a division operation by the division function 10e, perform a display operation by the display control function 10f, and perform a generation operation by the generation function 10g.

Thus, the calculation function 10a, the selection function 10b, the control function 10c, the division determination function 10d, the division function 10e, the display control function 10f, and the generation function 10g may respectively correspond to a calculation unit (or calculation logic), a sorting unit (selection unit or sorter), a control unit (or control logic), a division determination unit, a division unit, a display control unit, and a generation unit. Details about processing of each function of the processing circuit 10 will be described later. The control function 10c may also be an example of a transmission unit (or transmitter) having a function of transmitting various kinds of information.

The storage unit 20 is a storage region for storing instructions, data, and/or other information. Examples of the storage unit 20 include one or more of a random access memory (RAM), a read only memory (ROM), a flash memory, or a hard disk. In one embodiment, the storage unit may store various computer programs executed by the processing circuit 10 (e.g., to perform the aforementioned functions), execution results of the various computer programs, and the like.

The display 30 displays various kinds of information. For example, the display 30 displays a processing result of a CPU, a graphical user interface (GUI) for receiving various operations from a user, and the like. The display 30 is configured by a liquid crystal display, an organic Electro Luminescence (EL) display, or a cathode-ray tube display, for example. For example, the input device 40 and the display 30 may be integrally configured in a form of a touch panel.

The input device 40 is a device with which the user performs various operations. The input device 40 is configured by a mouse, a keyboard, a touch panel, or a hardware key, for example.

The embodiment is not limited to the examples described above. In some embodiments, the storage unit 20, the display 30, the input device 40, and the like are not essential components of the classical calculator 100. That is, the classical calculator 100 does not necessarily include the storage unit 20, the display 30, the input device 40, and the like, but rather may be coupled to these features. As another example, the storage unit 20, the display 30, the input device 40, and the like may be disposed outside the classical calculator 100 via a network, for example, and may exchange data with the processing circuit 10. In one embodiment, the classical calculator 100 may be configured by two or more classical calculators. For example, the classical calculator may include a first classical calculator having the calculation function 10a, and a second classical calculator that is connected to the first classical calculator via a network and has a sorting function (the selection function 10b) and the control function 10c as a transmission unit.

While the classical calculator 100 is a calculator for performing calculation to control the other calculators, each of the classical calculator 200a, the classical calculator 200b, the classical calculator 200c . . . is a calculator for performing given processing itself. An example of the given processing is a quantum chemistry calculation, but the given processing may be other types of processing (e.g., any type involving calculation) in other embodiments. For example, similarly to the classical calculator 100, the classical calculator 200a includes a processing circuit 10x similar to the processing circuit 10, and a storage unit 20x similar to the storage unit 20. The processing circuit 10x does not necessarily have all of the functions of the processing circuit 10. Similarly, each of the classical calculators 200b, 200c . . . also includes a processing circuit similar to the processing circuit 10 and a storage unit similar to the storage unit 20.

Each of the quantum calculators 300a, 300b, and 300c performs calculation by using a quantum state. For example, the quantum calculator 300a includes a quantum information generation unit (or quantum information generator) 50 that generates an initial quantum state, a plurality of quantum gates 60, a quantum gate control unit (or quantum gate controller) 70, and a measurement unit (measurer) 80.

The following describes a typical operation example of the quantum calculator. The processing circuit 10 transmits a control signal for generating a quantum state as an initial state to the quantum calculator 300a by the control function 10c as a transmission unit (or transmitter). After acquiring the control signal for generating the quantum state as the initial state from the classical calculator 100, the quantum calculator 300a generates an initial quantum state such as a quantum entanglement state, for example, by the quantum information generation unit 50.

The quantum gate 60 acts on the generated quantum state to change the quantum state. The action of the quantum gate 60 on the quantum state can be represented in a form of a unitary matrix. The quantum gate control unit 70 changes an operation of the quantum gate 60, that is, the unitary matrix. The measurement unit 80 measures the quantum state that has passed through the quantum gate 60. Due to this, the quantum calculator 300a can obtain, as output data, a measurement result measured by the measurement unit 80.

The calculator system 1 may also include a user terminal 400 that receives information input by the user, or displays information to the user. By way of example, similarly to the display 30 or the input device 40, the user terminal 400 may include a display 302 and an input device 40Z on the user terminal side.

Subsequently, the following briefly describes a background according to the embodiment.

A quantum calculator performs calculations under a calculation paradigm different from a classical calculator. For example, a quantum calculator may cause a unitary matrix to act on the quantum state (such as a quantum entangled state) that is initially generated under a predetermined quantum algorithm. Examples include Shore's prime factorization algorithm and Grover's search algorithm. A calculation result may then be generated by measuring the generated quantum state.

Simulation and execution environments of quantum calculation are provided as cloud services, such as Azure (registered trademark) Quantum, AWS (registered trademark) Braket, and IBM Q-Experience (registered trademark), for example. In these cloud services, for example, a test and simulation environment for a quantum algorithm on a classical computer is provided, and a created quantum algorithm can be executed on quantum hardware such as a gate-type superconducting computer, a quantum annealing superconducting computer, or an ion trap computer.

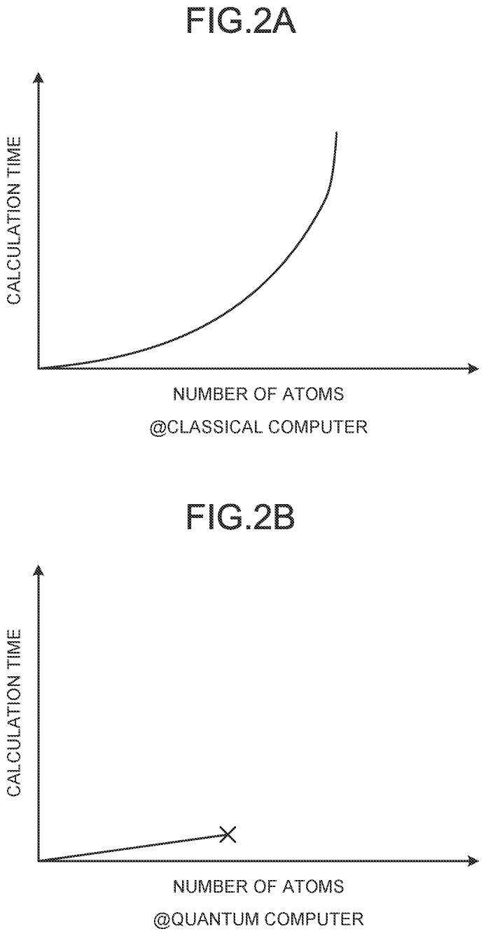

The following describes a difference between a quantum calculator and a classical calculator when performing giving processing for an example calculation, such as molecular orbital calculation. As illustrated in FIG. 2A, with the classical calculator, a calculation time abruptly increases as the number of atoms N increases. For example, the calculation time increases in proportion to N6 with respect to the number of atoms N.

On the other hand, as illustrated in FIG. 2B, with the quantum calculator, the calculation time does not increase so much with respect to the number of atoms N. For example, the calculation time is suppressed to be a time proportional to N with respect to the number of atoms N. However, it is difficult to make a quantum calculator having a large number of quantum bits under present circumstances, and there is a limitation by the number of quantum bits. Thus, in some circumstances one of the classical calculator or the quantum calculator may be selected to perform calculations depending on the amount of processing to be performed.

Considering the difficulty of making the quantum calculator have a large number of quantum bits, a combined algorithm may be used for performing calculations using both a classical algorithm executed by a classical calculator and a quantum algorithm executed by a quantum calculator. Such a combined algorithm may be used instead of performing all calculations by a quantum algorithm executed by the quantum calculator.

Examples of the combined algorithm include Variational Quantum Eigensolver (VQE). A VQE is an algorithm for measuring an expected value of Hamiltonian after generating a predetermined trial wave function by the quantum calculator based on a calculus of variations. In implementing the VQE, processing by the classical calculator may be performed based on a measurement result to embed the quantum calculator in the classical calculator based on the calculus of variations.

In addition to a case of using the combined algorithm that uses both of the classical calculator and the quantum calculator, there may be a case in which given processing is divided into processing performed by the classical calculator and processing performed by the quantum calculator. In such a case, the calculators may perform respective portions of the processing to thereby perform a parallel calculation. In a case in which the given processing is separately performed by a plurality of quantum calculators where the numbers of quantum bits are different from each other, there may be a case in which the given processing is divided into portions of processing performed by the respective quantum calculators, and the respective calculators may perform the portions of processing to perform the parallel calculation.

Embodiments described herein may be based on such a background, and in some cases aims at optimizing processing in a case of performing the processing by a plurality of calculators, including at least one quantum calculator.

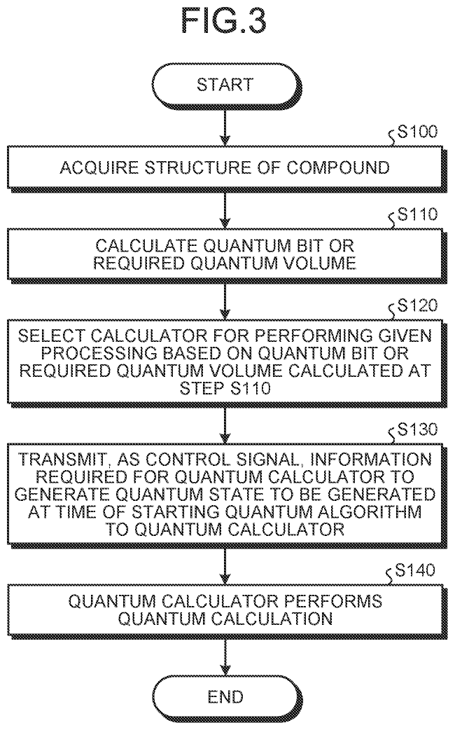

FIG. 3 is a flowchart illustrating a processing procedure performed by a computer program according to the first embodiment. The following describes performing molecular orbital calculation for a compound as an example of given calculation processing.

First, at Operation S100, the processing circuit 10 acquires information (such as a structure of the compound) from the storage unit, and makes a preparation for performing given processing, which may include, for example, processing of performing quantum chemistry calculation for a given chemical structure. That is, the processing circuit 10 assumes, as the given chemical structure, a chemical structure read out from the storage unit 20 storing a list of chemical structures, and makes a preparation for performing processing of performing quantum chemistry calculation.

Subsequently, at Operation S110, the processing circuit 10 calculates a quantum bit (qubit) or a quantum volume by the calculation function (or calculation logic) 10a. That is, at Operation S110, the computer program according to the embodiment causes the classical calculator 100 to perform a calculation operation of calculating a quantum bit or a quantum volume for performing the given processing, for example, the processing of performing quantum chemistry calculation on the given chemical structure by using the quantum algorithm, or to be used for a portion of the quantum algorithm in a case of performing the given processing using the combined algorithm. In one embodiment, the quantum volume may represent a quantum volume that is used, e.g., a scale of quantum calculation that can be performed, that is, a spatial size and a time. The quantum volume may be an indicator calculated by adding a size of an error to be caused to a simple number of quantum bits.

In calculating the quantum bit or the quantum volume calculated by the processing circuit 10 at Operation S110, the processing circuit 10 may calculate, or preferably calculates a value of the quantum bit or the quantum volume with a slight margin by the calculation function 10a. Herein, the margin can be optionally set in accordance with a type of calculation, a calculation speed, capacity of the calculator, a congestion state of a queue, and the like.

As a method of calculating the quantum bit or the quantum volume, for example, the processing circuit 10 calculates, by the calculation function 10a, the quantum bit or the quantum volume based on the number of atoms and a table representing a relation between the number of atoms and the quantum bit or the quantum volume for performing molecular orbital calculation of the number of atoms. The table is made, for example, by extrapolating a relational expression between the number of atoms and the quantum bit or the volume in a case in which the number of atoms is small such that a calculation amount can be accurately estimated to a case in which the number of atoms is large.

Subsequently, at Operation S120, in a case of performing the given processing by using the quantum algorithm, or by using the combined algorithm of the classical algorithm and the quantum algorithm, or by using both of the classical algorithm and the quantum algorithm, the computer program according to the first embodiment causes the classical calculator 100 to perform, by the selection function (or sorter) 10b, a selection operation of selecting the calculator for performing the given processing. Specifically, the processing circuit 10 selects, by the selection function 10b, the calculator for performing the given processing based on the quantum bit or the quantum volume calculated at Operation S110.

Herein, in a case in which the calculator as a candidate for performing the processing is a plurality of the quantum calculators, the selection operation of selecting the calculator for performing the given processing is an operation of selecting, from among the quantum calculators, the quantum calculator for performing the given processing based on the quantum bit or the quantum volume calculated at Operation S110.

In a case in which the calculator as a candidate for performing the processing is one or more quantum calculators and one or more classical calculators, the selection operation of selecting the calculator for performing the given processing is an operation of selecting, from the calculators including both of the classical calculator and the quantum calculator, the calculator for performing the given processing based on the quantum bit or the quantum volume calculated at Operation S110. In this case, by way of example, if the quantum bit or the quantum volume falls within a range of the quantum bit or the quantum volume that can be implemented by the quantum calculator as the candidate, the processing circuit 10 selects, by the selection function 10b, the quantum calculator as the calculator for performing the given processing. If the quantum bit or the quantum volume exceeds the range of the quantum bit or the quantum volume that can be implemented by the quantum calculator as the candidate, the processing circuit 10 selects, by the selection function 10b, the classical calculator as the calculator for performing the given processing.

For example, in a case in which the number of quantum bits of the quantum calculator 300a is 100 quantum bits, and the number of quantum bits calculated at Operation S110 is 80 quantum bits, at Operation S120, the processing circuit 10 selects, by the selection function 10b, the quantum calculator 300a as the calculator for performing the given processing. The quantum calculator 300a performs the given processing by using the quantum algorithm.

On the other hand, in a case in which the number of quantum bits of the quantum calculator 300a is 100 quantum bits, and the number of quantum bits calculated at Operation S110 is 120 quantum bits, at Operation S120, the processing circuit 10 selects, by the selection function 10b, the classical calculator 200a as the calculator for performing the given processing. In a case in which the given processing is molecular orbital calculation, the classical calculator 200a performs molecular orbital calculation by using a Hartree-Fock method, a DFT method, or a CCSD (T) method, for example.

In a case of using the combined algorithm of the classical algorithm and the quantum algorithm, the selection operation of selecting the calculator for performing the given processing is an operation of selecting the quantum calculator for performing the given processing based on the quantum bit or the quantum volume calculated at Operation S110, and selecting the classical calculator for executing the classical algorithm separately from the selection of the quantum calculator.

At these selection operations, the processing circuit 10 selects the quantum calculator by the selection function 10b so that the quantum bit calculated at Operation S110 does not exceed the quantum bit that can be used by the calculator to be selected, or the quantum volume calculated at Operation S110 does not exceed the quantum volume that can be implemented by the calculator to be selected.

At the selection operation, in a case in which the system includes a plurality of the quantum calculators the quantum bits or quantum volumes of which are different from each other, it is preferable to preferentially select the quantum calculator having the quantum bit or volume that is not smaller than and is closest to the quantum bit or the quantum volume to be used for calculation of a target compound at the selection operation.

At the selection operation, the processing circuit 10 may select, by the selection function 10b, the calculator while considering capacity, a characteristic, a vacant state, and the like of an available calculator resource in addition to the calculated quantum bit or quantum volume. By way of example, the processing circuit 10 selects, by the selection function 10b, the calculator having high capacity of the calculator resource and having a characteristic appropriate for the given processing, or a relatively vacant calculator as the calculator for performing the processing.

At the selection operation, the processing circuit 10 may select, by the selection function 10b, not only the calculator but also an execution timing or execution order for executing the calculator.

In addition to the processing performed at Operation S120, the processing circuit 10 appropriately displays, by the display control function 10f, the quantum bit or the quantum volume calculated by the calculation function 10a, information about the calculator selected by the selection function 10b, and a character string representing a structural formula or atomic coordinates or a chemical structure, or information specifying a compound on a display unit. Herein, the display unit may be the display 30, or may be the user terminal 400 as a terminal that can be operated by the user and is different from a terminal on the provider side that is a terminal on which the classical calculator 100 is disposed.

The processing circuit 10 may have a function of transmitting, by the control function 10c, the quantum bit or the quantum volume calculated by the calculation function 10a or the information about the calculator selected by the selection function 10b to the other calculator, the user terminal 400, a terminal external to the calculator system 1, or the like.

Subsequently, at Operation S130, in a case in which the calculator selected at Operation S120 includes the quantum calculator, the computer program according to the embodiment causes, by the selection function 10b as a transmission unit (or transmitter), the classical calculator 100 to perform a control operation of generating a control signal to be transmitted to the quantum calculator. (In some embodiments, the classical calculator 100 may be considered to be or include control logic). Herein, the control signal may be a control signal related to an instruction by which the quantum calculator starts the quantum algorithm, for example, information for the quantum calculator to generate a quantum state to be generated at the time of starting the quantum algorithm.

For example, assuming that the quantum state generated at the time of starting the quantum algorithm can be represented as α1|0>+α2|1>, coefficients α1 and α2 are information for the quantum calculator to generate the quantum state α1|0>+α2|1> to be generated at the time of starting the quantum algorithm. Subsequently, the processing circuit 10 transmits, by the control function 10c as a transmission unit, the information for the quantum calculator to generate the quantum state to be generated at the time of starting the quantum algorithm to the quantum calculator as the control signal.

Subsequently, at Operation S140, the quantum calculator performs quantum calculation based on the control signal acquired from the classical calculator 100. In a case of performing calculation by the classical calculator, the classical calculator performs normal calculation based on the control signal acquired from the classical calculator 100. In a case of performing calculation by using the combined algorithm of calculation by the classical calculator and calculation by the quantum calculator, the classical calculator and the quantum calculator perform calculation based on the control signal acquired from the classical calculator 100. The classical calculator 100 transmits, by the control function 10c serving as a transmission unit, the control signal to a plurality of the calculators, and each of the calculators performs each piece of the processing based on the control signal received by each of the calculators.

The following embodiment describes a case in which the classical calculators 200a, 200b, 200c . . . and the quantum calculators 300a, 300b, 300c . . . perform molecular orbital calculation by using a Variational Quantum Eigensolver (VQE) method as the combined algorithm of the classical algorithm and the quantum algorithm.

The VQE method is a method using a variation principle in which a variation parameter is assumed to be θ and an expected value <ψ(θ)|H|ψ(θ)> related to a trial wave function ψ(θ) with respect to Hamiltonian H of a system is always larger than energy E in a ground state, and incorporating calculation by the quantum calculator into an algorithm for performing molecular orbital calculation.

Herein, the quantum calculator 300a and the like generate the quantum state ψ(θ) as a target of the calculus of variations with respect to the certain variation parameter θ as the quantum state using the quantum algorithm. The quantum calculator 300a and the like also represent, on the quantum calculator, the Hamiltonian H of the system that performs molecular orbital calculation and the like as Hamiltonian with respect to the quantum state ψ(θ) generated on the quantum calculator by the quantum information generation unit 50. Thereafter, the measurement unit 80 measures the expected value of the Hamiltonian <ψ(θ)|H|ψ(θ)> with respect to the quantum state ψ(θ) generated on the quantum calculator. Due to this, the quantum calculator 300a can obtain, as a measurement value, the expected value of the Hamiltonian H of the system in the generated quantum state ψ(θ) with the given variation parameter θ.

On the other hand, the classical calculator 200a and the like perform other part of the processing, for example, processing of selecting the variation parameter θ, processing of calculating the variation parameter θ with which the expected value of Hamiltonian becomes the smallest, processing of determining to select which quantum state ψ(θ), based on a value of the variation parameter θ, and the like, and optimize the variation parameter θ. Due to this, the classical calculator 200a and the like and the quantum calculator 300a and the like can perform quantum chemistry calculation and the like in cooperation with each other.

As described above, with the computer program according to the first embodiment, in a case in which the classical calculator and the quantum calculator are present, or a case in which a plurality of the quantum calculators are present, it is possible to appropriately select the calculator for performing the processing, and as a result, the processing can be optimized.

First Modification of First Embodiment

The first embodiment describes a case in which the computer program automatically selects the calculator for performing the given processing based on the quantum bit or the quantum volume calculated at the calculation operation. The embodiment is not limited thereto. In a first modification of the first embodiment, the computer program may cause the display 30 to display the calculated quantum bit or quantum volume, and may receive feedback from the user who has viewed the quantum bit or the quantum volume displayed on the display 30.

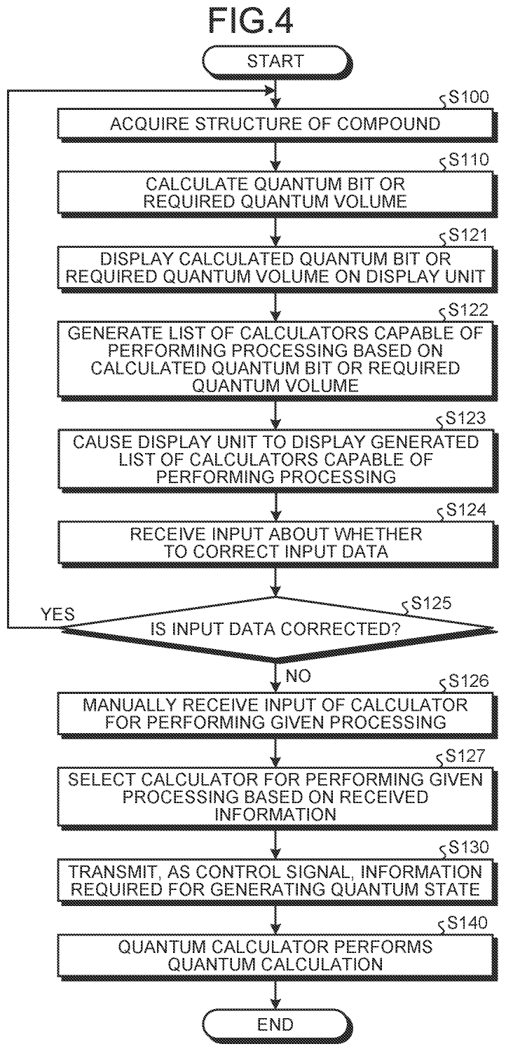

Such a situation is illustrated in FIG. 4. The processing at Operation S100, Operation S110, Operation S130, and Operation S140 may be the same as the processing that has been already described above with reference to FIG. 2, so that redundant description will not be repeated.

At Operation S121, the computer program according to the first modification of the first embodiment causes, by the display control function 10f of the processing circuit 10, the classical calculator 100 to further perform a display control operation of causing the display unit to display the quantum bit or the quantum volume calculated at the calculation operation S110.

Subsequently, at Operation S122, the computer program according to the first modification of the first embodiment causes, by the processing circuit 10, the classical calculator 100 to perform a generation operation of generating a list of calculators that can perform the processing based on the quantum bit or the quantum volume calculated at Operation S121.

Subsequently, at Operation S123, the computer program causes, by the display control function 10f of the processing circuit 10, the classical calculator 100 to perform a display control operation of causing the display unit to display the list of calculators that can perform the processing generated at Operation S122.

Subsequently, at Operation S124, the computer program causes, by the control function 10c of the processing circuit 10, the classical calculator 100 to receive an input about whether to correct input data such as a compound name from the user.

If the processing circuit 10 receives, by the control function 10c, the input for correcting the input data from the user (Yes at Operation S125), the process returns to Operation S100. On the other hand, if the input data is not corrected (No at Operation S125), the process proceeds to Operation S126.

Subsequently, at Operation S126, the computer program according to the first modification of the first embodiment causes the classical calculator 100 to perform, by the control function 10c of the processing circuit 10, an operation of manually receiving an input of the calculator for performing the given processing.

Subsequently, at Operation S127, the computer program according to the first modification of the first embodiment causes the classical calculator 100 to perform, by the selection function 10b of the processing circuit 10, an operation of selecting the calculator for performing the given processing based on the input received from the user at Operation S126.

Similarly to the first embodiment, the computer program according to the first modification of the first embodiment causes the classical calculator 100 to perform the same processing as the processing at Operation S130 and Operation S140.

As described above, with the computer program according to the first embodiment, in a case in which the classical calculator and the quantum calculator are present, or a case in which a plurality of the quantum calculators are present, it is possible to appropriately select the calculator for performing the processing by receiving the input from the user as needed, and as a result, the processing can be optimized.

### Second Embodiment

The first embodiment describes a case in which one calculator performs the entire processing without dividing the given processing into a plurality of small portions of processing. A second embodiment describes a case in which the given processing is divided into a plurality of smaller portions of processing, and the classical calculator or the quantum calculator performs the processing by the calculator selected for each of the smaller portions of processing.

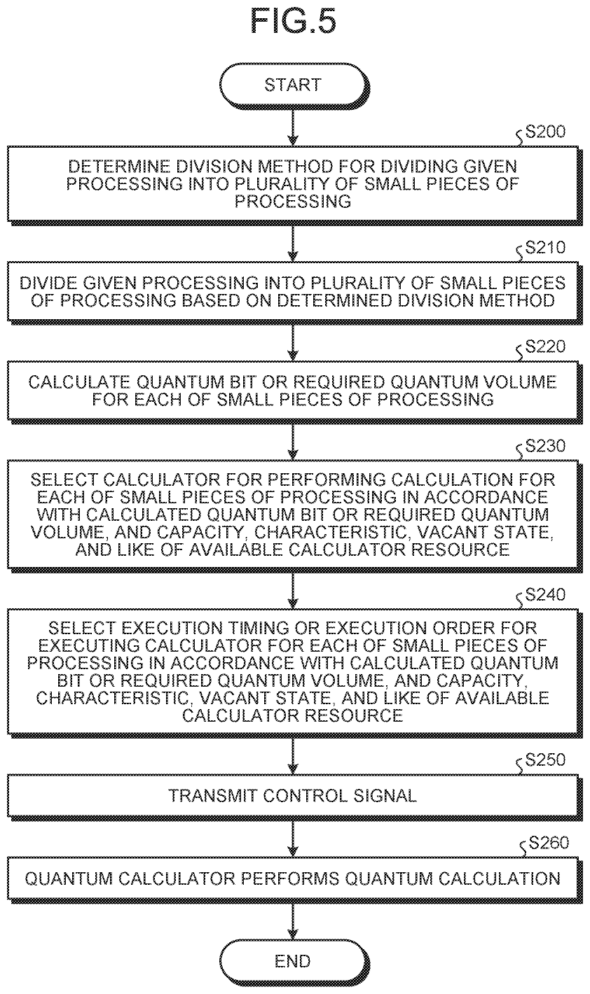

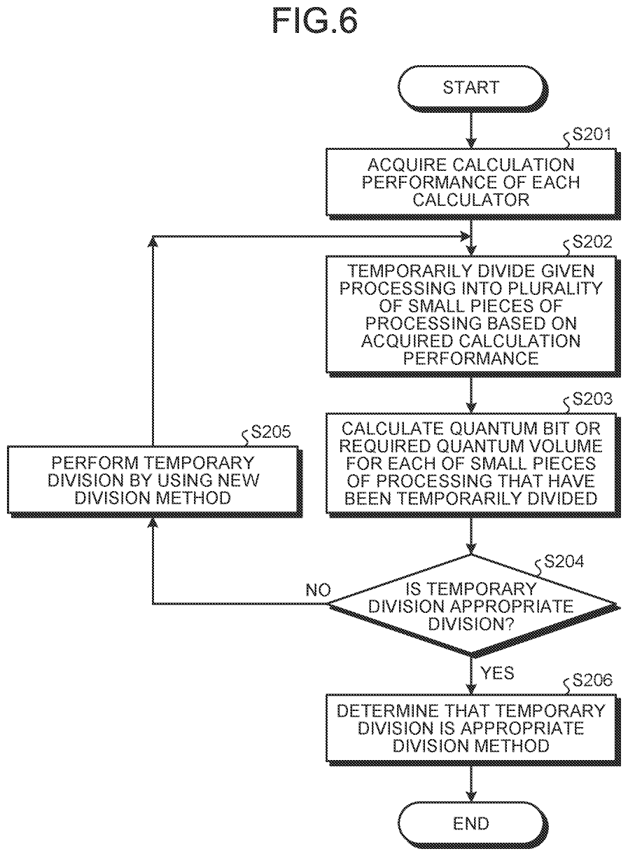

Such a situation is illustrated in FIG. 5 and FIG. 6. FIG. 5 is a flowchart illustrating a processing procedure that is caused to be performed by the classical calculator 100 by a computer program according to the second embodiment, and FIG. 6 is a flowchart for specifically explaining processing at Operation S200 in FIG. 5.

First, at Operation S200, the computer program according to the second embodiment causes the classical calculator 100 to perform, by a division determination function 10d of the processing circuit 10, a division determination operation of determining a division method for dividing the given processing into a plurality of processing portions.

The following specifically describes the division determination operation at Operation S200 with reference to FIG. 6. The entire processing in FIG. 6 corresponds to Operation S200 in FIG. 5.

First, at Operation S201, the computer program according to the second embodiment causes the classical calculator 100 to acquire calculation performance of each calculator.

Subsequently, at Operation S202, the computer program according to the second embodiment causes the classical calculator 100 to perform, by the division determination function 10d of the processing circuit 10, an operation of temporarily dividing the given processing into a plurality of processing portions based on the calculation performance acquired at Operation S201.

Subsequently, at Operation S203, the computer program according to the second embodiment causes the classical calculator 100 to perform an operation of calculating the quantum bit or quantum volume for each of the processing portions that have been temporarily divided.

Subsequently, if the temporary division satisfies a predetermined condition and is appropriate division (Yes at Operation S204), the process proceeds to Operation S206, the processing circuit 10 determines that the temporary division is an appropriate division method, and the processing circuit 10 determines that the division is an appropriate division method.

On the other hand, if the temporary division is not appropriate division (No at Operation S204), the computer program according to the second embodiment causes the classical calculator 100 to perform an operation of performing temporary division using a new division method at Operation S205, and the process returns to Operation S202 thereafter.

Through the portions of processing at Operation S201 to Operation S206, the computer program according to the second embodiment causes the classical calculator 100 to perform the processing related to Operation S200 described above.

Subsequently, returning to FIG. 5, at Operation S210, the computer program according to the second embodiment causes the classical calculator 100 to perform, by the division function 10e of the processing circuit 10, an operation of dividing the given processing into a plurality of processing portions based on the division method determined at Operation S200.

In the embodiment, the given processing may be constituted of a plurality of processing portions without performing the processing at Operation S200 and Operation S210. In such a case, operations at Operation S200 and Operation S210 are omitted as needed.

Subsequently, at Operation S220, the computer program according to the second embodiment causes the classical calculator 100 to perform, by the calculation function 10a of the processing circuit 10, a calculation operation of calculating the quantum bit or quantum volume for each of the processing portions.

Subsequently, at Operation S230, the computer program according to the second embodiment causes the classical calculator 100 to perform a selection operation of selecting the calculator for performing calculation for each of the processing portions in accordance with the calculated quantum bit or quantum volume, and capacity, a characteristic, a vacant state, and the like of an available calculator resource. For example, the computer program according to the second embodiment causes the classical calculator 100 to preferentially select, as the calculator for performing calculation, a calculator having high calculation capacity, a calculator the characteristic of which matches a property of the given processing, and a relatively vacant calculator.

Subsequently, at Operation S240, the computer program according to the second embodiment causes the classical calculator 100 to further select, by the selection function 10b of the processing circuit 10, an execution timing or execution order for executing the calculator for each of the processing portions in accordance with the calculated quantum bit or quantum volume, and capacity, a characteristic, a vacant state, and the like of an available calculator resource. By way of example, the computer program according to the second embodiment causes the execution timing or execution order for executing the calculator to be selected so that a processing load of the calculator does not concentrate on a specific calculator.

Subsequently, at Operation S250, the computer program according to the second embodiment causes the classical calculator 100 to perform, by the control function 10c of the processing circuit 10, a control operation of generating, as a control signal, information to be used for the quantum calculator to generate the quantum state to be generated at the time of starting the quantum algorithm.

The processing circuit 10 transmits, by the control function 10c as a transmission unit, the generated control signal to the calculator selected at Operation S230.

Subsequently, at Operation S260, the quantum calculator performs quantum calculation.

As described above, with the computer program according to the second embodiment, in a case in which the classical calculator and the quantum calculator are present, or a case in which a plurality of the quantum calculators are present, when the given processing is constituted of a plurality of processing portions, the processing can be optimized by appropriately dividing the processing and appropriately selecting the calculator for performing the processing for each divided portion of the processing.

According to one or more embodiments of the present invention, the processing can be optimized in a case in which the classical calculator and the quantum calculator are present, or a case in which a plurality of the quantum calculators are present. Moreover, according to one or more embodiments of the present invention, processing can be optimized (or improved) in a case in which a classical calculator and a quantum calculator are present, or a case in which a plurality of quantum calculators are present.

The methods, processes, and/or operations described herein may be performed by code or instructions to be executed by a computer, processor, controller, or other signal processing device. The computer, processor, controller, or other signal processing device may be those described herein or one in addition to the elements described herein. Because the algorithms that form the basis of the methods (or operations of the computer, processor, controller, or other signal processing device) are described in detail, the code or instructions for implementing the operations of the method embodiments may transform the computer, processor, controller, or other signal processing device into a special-purpose processor for performing the methods herein.

Also, another embodiment may include a computer-readable medium, e.g., a non-transitory computer-readable medium, for storing the code or instructions described above. The computer-readable medium may be a volatile or non-volatile memory or other storage device, which may be removably or fixedly coupled to the computer, processor, controller, or other signal processing device which is to execute the code or instructions for performing the method embodiments or operations of the apparatus embodiments herein.

The controllers, processors, devices, circuits, functions, calculators, gates, dividers, modules, units, generators, logic, interfaces, and other signal generating and signal processing features of the embodiments disclosed herein may be implemented, for example, in non-transitory logic that may include hardware, software, or both. When implemented at least partially in hardware, the controllers, processors, devices, circuits, functions, calculators, gates, dividers, modules, units, generators and other signal generating and signal processing features may be, for example, any one of a variety of integrated circuits including but not limited to an application-specific integrated circuit, a field-programmable gate array, a combination of logic gates, a system-on-chip, a microprocessor, or another type of processing or control circuit.

When implemented in at least partially in software, the controllers, processors, devices, circuits, functions, calculators, gates, dividers, modules, units, generators and other signal generating and signal processing features may include, for example, a memory or other storage device for storing code or instructions to be executed, for example, by a computer, processor, microprocessor, controller, or other signal processing device. The computer, processor, microprocessor, controller, or other signal processing device may be those described herein or one in addition to the elements described herein. Because the algorithms that form the basis of the methods (or operations of the computer, processor, microprocessor, controller, or other signal processing device) are described in detail, the code or instructions for implementing the operations of the method embodiments may transform the computer, processor, controller, or other signal processing device into a special-purpose processor for performing the methods described herein.

Although the invention has been described with respect to specific embodiments for a complete and clear disclosure, the appended claims are not to be thus limited but are to be construed as embodying all modifications and alternative constructions that may occur to one skilled in the art that fairly fall within the basic teaching herein set forth.

What is claimed is:

1. A method for performing given processing using a quantum algorithm or a combined algorithm which comprises a classical algorithm and the quantum algorithm, the method comprising:
calculating a quantum bit or a quantum volume for performing the given processing using the quantum algorithm, or for the quantum algorithm when the given processing is to be performed using the combined algorithm;
selecting a calculator from a plurality of calculators for performing the given processing based on the quantum bit or the quantum volume; and
generating a control signal when the selected calculator includes a quantum calculator, wherein the control signal is to be transmitted to the quantum calculator and corresponds to an instruction by which the quantum calculator starts the quantum algorithm.

2. The method according to claim 1, wherein the control signal corresponds to information for the quantum calculator to generate a quantum state at a time of starting the quantum algorithm.

3. The method according to claim 1, wherein the given processing comprises performing a quantum chemistry calculation on a given chemical structure.

4. The method according to claim 3, wherein the given chemical structure is read out from a storage unit storing a list of chemical structures.

5. The method according to claim 1, wherein
the given processing comprises a plurality of processing portions,
the quantum bit or the quantum volume is calculated for each of the processing portions,
the calculator is selected for each of the processing portions, and
the control signal is generated for each of the processing portions.

6. The method according to claim 1, further comprising:
a division determination operation to determine a division method for dividing the given processing into a plurality of processing portions; and
a division operation to divide the given processing into the processing portions based on the determined division method, wherein
the calculator is selected for each of the processing portions divided at the division operation, and
the control signal is generated for each of the processing portions divided at the division operation.

7. The method according to claim 6, wherein the calculator is selected in accordance with the calculated quantum bit or the quantum volume and at least one selected from the group consisting of capacity, a characteristic, and a vacant state of an available calculator resource.

8. The method according to claim 7, further comprising:
selecting an execution timing or execution order for executing the calculator in accordance with the quantum bit or the quantum volume and at least one selected from the group consisting of capacity, a characteristic, and a vacant state of an available calculator resource.

9. The method according to claim 1, wherein the calculator is selected so that the quantum bit does not exceed a quantum bit that is able to be used by the calculator or the quantum volume does not exceed a quantum volume of the calculator.

10. The method according to claim 1, further comprising: a display control operation to cause a display to display the calculated quantum bit or the quantum volume.

11. The method according to claim 1, further comprising:
generating a list of calculators capable of performing the given processing based on the calculated quantum bit or the quantum volume; and
controlling a display to display the list of calculators capable of performing the given processing.

12. The method according to claim 10, wherein the display is disposed on a terminal different from a terminal on a provider side that comprises the classical calculator, and wherein the different terminal is able to be operated by a user.

13. The method according to claim 1, wherein the selected calculator is selected from both of the classical calculator and the quantum calculator based on the quantum bit or the quantum volume.

14. The method according to claim 1, wherein the combined algorithm is a variational quantum eigensolver (VQE) method.

15. A calculator system, comprising:
a plurality of calculators; and
a classical calculator configured to select a calculator for performing given processing from among the plurality of calculators, the given processing to be performed using a quantum algorithm or a combined algorithm of a classical algorithm and the quantum algorithm, wherein:
the calculators include at least one quantum calculator,
the classical calculator comprises:
calculation logic configured to calculate a quantum bit or quantum volume for performing the given processing using the quantum algorithm, or for the quantum algorithm in a case of performing the given processing using the combined algorithm;
a sorter configured to select the calculator for performing the given processing based on the quantum bit or the quantum volume; and
a transmitter configured to transmit an instruction to the quantum calculator when the calculator selected by the sorter includes the quantum calculator, the instruction being one by which the quantum calculator starts the quantum algorithm.

16. The calculator system according to claim 15, comprising:
logic configured to transmit the calculated quantum bit or the quantum volume or information related to the selected calculator.

17. The calculator system according to claim 15, further comprising:
a display configured to display the calculated quantum bit or the quantum volume or information related to the selected calculator.

18. The calculator system according to claim 17, further comprising:
a display configured to display the calculated quantum bit or the quantum volume or the information related to the selected calculator and a character string representing a structural formula or atomic coordinates or a chemical structure or information specifying a compound.

19. The calculator system according to claim 15, wherein the classical calculator comprises:
a first classical calculator configured to perform an operation of the calculation logic, and
a second classical calculator connected to the first classical calculator via a network and configured to perform operations of the sorter and the transmitter.

20. A classical calculator for performing given processing using a quantum algorithm or a combined algorithm which comprises a classical algorithm and the quantum algorithm, the classical calculator comprising:
calculation logic configured to calculate a quantum bit or a quantum volume for performing the given processing using the quantum algorithm, or for the quantum algorithm in a case of performing the given processing using the combined algorithm;
a sorter configured to select a calculator from a plurality of calculators, the selected calculated to perform the given processing based on the calculated quantum bit or the quantum volume calculated by the calculation logic; and
a transmitter configured to transmit an instruction to a quantum calculator when the selected calculator includes the quantum calculator, the instruction being one by which the quantum calculator starts the quantum algorithm.
